(19) United States
(12) Patent Application Publication
(10) Pub. No.: US 2024/0245902 A1
(43) Pub. Date: Jul. 25, 2024

(54) METHOD AND APPARATUS FOR MANUFACTURING A CARDIAC SUPPORT SYSTEM

(71) Applicant: KARDION GMBH, Stuttgart (DE)

(72) Inventors: Thomas Alexander Schlebusch, Stuttgart (DE); David Minzenmay, Stuttgart (DE); Julian Kassel, Stuttgart (DE); Ingo Stotz, Stuttgart (DE)

(21) Appl. No.: 18/694,363

(22) PCT Filed: Sep. 22, 2022
(86) PCT No.: PCT/US2022/076885

§ 371 Date: Mar. 21, 2024

Related U.S. Application Data
(60) Provisional application No. 63/261,552, filed on Sep. 23, 2021.

(51) Int. Cl. A61M 60/139; A61M 60/237; A61M 60/411; A61M 60/816; A61M 60/859; A61M 60/861

(52) U.S. Cl. CPC A61M 60/139; A61M 60/237; A61M 60/411; A61M 60/816; A61M 60/859; A61M 60/861; A61M 2205/103; A61M 2205/3327; A61M 2205/3334; A61M 2205/3368; A61M 2207/10; A61M 2210/127

(57) ABSTRACT

Systems and methods relating to manufacturing and assembling a cardiac support system. The method may include a step of providing a sensor device and an inlet tube. The inlet tube may be adapted to aspirate a body fluid of a patient and may include a drive unit for operating the cardiac support system. The method may further include a step of connecting the sensor device to a first end of the inlet tube and the drive unit to a second end of the inlet tube.

### RELATED APPLICATIONS

The present application claims the benefit of U.S. Provisional Application No. 63/261,552, filed Sep. 23, 2021, titled METHOD AND APPARATUS FOR MANUFACTURING A CARDIAC ASSIST SYSTEM, the entire contents of which are incorporated by reference herein and made a part of this specification for all purposes.

### BACKGROUND

Disclosed herein are methods and devices for manufacturing a cardiac support system, and a corresponding computer program. Cardiac support systems are used in medicine, for example, to relieve a weakened heart of a patient. These systems may be heart pumps or mechanical circulatory support devices and may be configured to support or improve heart function of a patient. These systems may support heart function via the coronary arteries.

### SUMMARY

The approach presented here relates to improved methods for manufacturing a cardiac support system, improved cardiac support systems, devices using this method, and a corresponding computer program.

The approach presented here enables reduction of required production space of a cardiac support system by advantageously providing a production and/or assembly sequence of individual components of the cardiac support system, while simultaneously ensuring functionality of the cardiac support system.

Methods for manufacturing a cardiac support system are presented, which may include provisioning steps and/or connecting steps. The provisioning steps may include providing and/or manufacturing a sensor device, an inlet tube with a first end and a second end opposite the first end, the inlet tube configured for aspirating a patient's body fluids, and a drive unit for operating the cardiac support system. In the connecting steps, the sensor device may be connected to the first end of the inlet tube and the drive unit may be connected to the second end of the inlet tube to create at least a portion of the cardiac support system.

The approach described herein may be advantageously used to create a biocompatible and long-term stable option, for example, to integrate a sensor cable into a cardiac support system. A sensor device can be advantageously configured to measure flow velocity and/or volume flow of a body fluid (e.g., blood), and/or characteristics of the body fluid such as pressure, viscosity, and/or temperature.

According to an example, a sensor device and an inlet tube may be connected in a connecting step to create a connecting device. Advantageously, the sensor device may be located at a distal tip of the connecting device to obtain data in an operational condition without disrupting pumping action of the cardiac support system. With this configuration, erroneous readings can be advantageously reduced. The connecting device may be connected to a drive unit in a connecting step to create at least a portion of the cardiac support system. Thus, the cardiac support system may advantageously be manufactured in a modular fashion.

According to an example, a housing element for the cardiac support system may be provided in a provisioning step. In a connecting step, the drive unit may be connected to the housing element. The housing element may be shaped, for example, to protect electrical components of the cardiac support system from external influences. Additionally, the housing element may be shaped to protect a blood vessel into which the cardiac support system is inserted, for example, from a pump element of the cardiac support system.

According to an example, a manufacturing method may include a step of pre-positioning and fixing a sensor device, an inlet tube, and/or a drive unit. The sensor device, the inlet tube and/or the drive unit may, in a fixing step, be bonded together, siliconized, and/or conductively bonded. At least a part of the pre-positioning and fixing step may be repeated as desired. It may be advantageous to connect subcomponents in a modular sequence to form result components by, for example, material bonding and/or electrically connecting. In other words, two sub-components may be combined in a modular fashion to form a result component, which, in turn, may be combined with at least one further sub-component to form a further result component. This advantageous procedure may be implemented in software, hardware and/or a mixed form of software and hardware, such as an automated control unit. The approach presented allows manufacture of a device that may be configured to perform, control, or implement alternative steps of the process presented here in appropriate facilities. For this purpose, the device may include at least one computing unit for processing signals or data, at least one memory unit for storing signals or data, at least one interface to a sensor or an actuator for reading in sensor signals from the sensor or for outputting data or control signals to the actuator and/or at least one communication interface for reading in or outputting data, which may be embedded in a communication protocol. The at least one computing unit may be, for example, a signal processor, a microcontroller, or the like, whereby the memory unit may be a flash memory, an EEPROM or a magnetic memory unit. The at least one communication interface may be configured to read data wirelessly and/or wired or whereby the at least one communication interface which can read in or output wired data may, for example, read this data electrically or optically from a corresponding data transmission line or output it to a corresponding data transmission line. In this case, a device may be understood to be an electrical device that processes sensor signals and, depending on these, outputs control and/or data signals. The device may have at least one interface, which may have hardware and/or software features. In the case of hardware, the at least one interface may be part of a so-called system ASICs, which may include various functions of the device. The at least one interface may include integrated circuits or at least partly include discrete components. In the case of software-based training, the at least one interface may be software modules, which can be installed on a microcontroller alongside other software modules that may be available.

A computer program product or computer program with program code that is stored on a machine-readable medium or storage medium such as a semiconductor memory, hard disk storage or optical memory and can be used for execution, conversion and/or control of the steps of the above-described procedures may be used, especially if the program product or program is executed on a computer or device.

Also presented is a cardiac support system including a sensor device, an inlet tube including a first end and a second end opposite the first end, connected to the sensor device, the inlet tube configured to aspirate a patient's body fluid, and a drive unit for operating the cardiac support system, the drive unit being connected to the second end of the inlet tube.

The sensor device may be configured, for example, to obtain measured values that are as precise and unaltered as possible. The inlet tube may be advantageously configured to conduct the body fluid, for example blood. The first end of the inlet tube may be connected to the sensor device.

According to an example, the inlet tube may have at least one inlet opening at or near the first end, which may be configured to allow at least one body fluid to flow into the inlet tube. The at least one inlet opening may be configured as an interface between the sensor device and the inlet tube. Advantageously, the interface may be configured as an inlet grille, which may form at least one inlet opening. Advantageously, the at least one inlet opening may include at least two inlet openings (e.g., two, three, four, five, six).

According to an example, the inlet tube may have at least one outlet opening at the second end, which may be configured to allow the at least one body fluid to be discharged from the inlet tube. Advantageously, the at least one body fluid may be conducted into a blood vessel in a regulated manner, thereby, for example, supporting heart function.

According to an example, the cardiac support system may have a deployable anchoring structure, a driver (e.g., motor), a pump element, and/or a connection interface. The deployable anchoring structure may be configured, for example, to advantageously anchor the cardiac support system in position. In one implementation, the deployable anchoring structure may have a grid-like structure, which can advantageously be connected (e.g., facilitate tissue ingrowth) to a tissue of the patient. The driver may be configured to drive, for example, the pump element. The pump element may be implemented as, for example, an impeller or a pump wheel. The connection interface may be configured to, for example, connect electrical conductors.

The sensor device may have a plurality of sensor components, including at least one sensor, layer substrate, carrier element and/or an ultrasonic transducer. Advantageously, the plurality of sensor components may be connected to each other in a modular fashion.

According to an example, at least two of the plurality of sensor components may be electrically connected to each other. Advantageously, for example, signals or an electrical voltage may be transmitted via the connection to the corresponding sensor components.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the drawings, can be arranged, substituted, combined, and designed in a wide variety of different configurations, all of which are explicitly contemplated and make part of this disclosure. Execution examples of the approach presented here are shown in the drawings and explained in more detail in the following description. It shows:

FIG. 1 is a schematic representation of a cardiac support system according to an example.

FIG. 2 is a schematic representation of a cardiac support system according to an example of an implanted state.

FIG. 3 is a flow chart of a manufacturing process for manufacturing a cardiac support system according to an example.

FIG. 4 is a block diagram of a device according to an example.

FIG. 5 is a production sequence for manufacturing a sensor device with a plurality of sensor components according to an example.

FIG. 6 is an assembly sequence for manufacturing a connecting device according to an example.

FIG. 7 is a further assembly sequence for connecting a connecting device to a drive unit according to an example.

FIG. 8 is a cabling sequence for wiring a cardiac support system according to an example.

### DETAILED DESCRIPTION

In the following description, examples of the present invention use identical or similar reference signs for elements represented in different figures and having a similar effect, without repeated description of these elements.

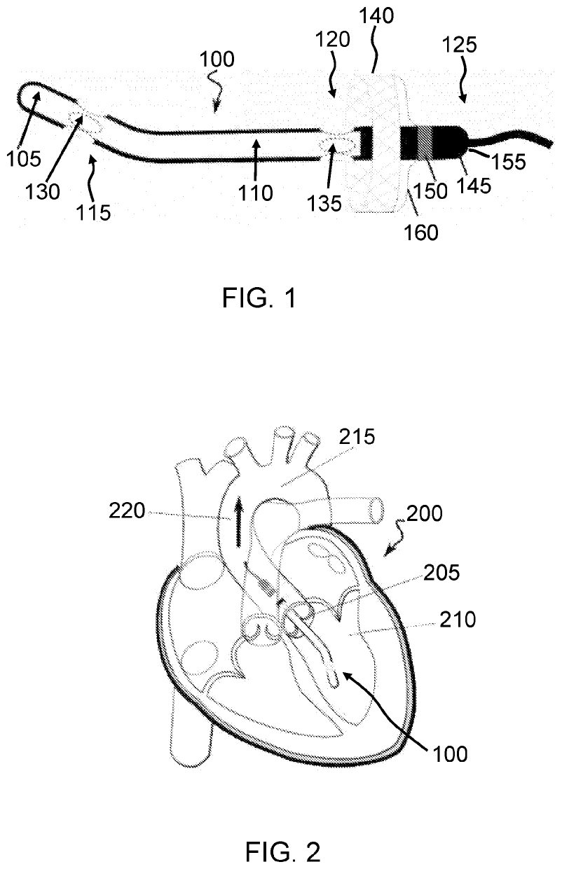

FIG. 1 shows a schematic representation of a cardiac support system 100 according to an example. The cardiac support system 100, for example, may be implemented as a heart pump and may be configured to support a patient's heart function. The cardiac support system 100 may include a sensor device 105, which may be configured, for example, to measure, for example, a flow velocity and/or volume flow of a body fluid and/or characteristics of the body fluid such as pressure, viscosity and/or temperature.

The cardiac support system 100 may include an inlet tube 110, with a first end 115 and a second end 120 opposite the first end 115, connected to the sensor device 105. The inlet tube 110 may be configured to aspirate a patient's body fluid such as, for example, blood. According to an example, the first end 115 may be connected to the sensor device 105. Furthermore, the cardiac support system 100 may include a drive unit 125 for operating the cardiac support system 100. The drive unit 125 may be connected to the second end 120 of the inlet tube 110. According to an example, a component 165 (not shown) may be positioned between the second end 120 of the inlet tube and the drive unit 125. The component 165 (not shown) may be configured as an interface between the inlet tube 110 and the drive unit 125. In an example, the component 165 may be implemented as an impeller housing. In an example, the component 165 (not shown) may include at least one outlet opening. The inlet tube 110 may be bent or at least curved between the first end 115 and the second end 120, for example, in a region closer to the first end 115. The bend or curve may be a rounded corner.

According to an example, the sensor device 105 may have a plurality of sensor components. The plurality of sensor components may be formed with at least one sensor, layer substrate, carrier element and/or an ultrasonic transducer, which may be electrically connected to each other. According to an example, the inlet tube 110 may have at least one inlet opening 130 at the first end 115. The at least one inlet opening 130 may be configured, for example, to allow at least one body fluid to flow into the inlet tube 110. The at least one inlet opening 130 may be configured, for example, as an inlet grille and may be configured as an interface between the sensor device 105 and the inlet tube 110. According to an example of the inlet tube 110, the at least one inlet opening 130 may include a plurality of inlet openings (e.g., at least three similarly shaped openings), which may be oval, rectangular, square, round, or rhomboid in shape. At the second end 120, the inlet tube 110 may include at least one outlet opening 135. According to an example, the at least one outlet opening 135 may be configured to allow discharge of the at least one body fluid from the inlet tube 110. As with the at least one inlet opening 130, the at least one outlet opening 135 may be oval, rectangular, square, round, or rhomboid in shape. The inlet tube 110 may be configured according to an example with the at least one outlet opening 135 including at least three outlet openings which, like the at least one inlet opening 130, may be configured, for example, as an outlet grille.

According to an example, the cardiac support system 100 may include a deployable anchoring structure 140, a drive unit 125, a pump element 150 and/or a connection interface 155. The deployable anchoring structure 140 may be configured, for example, to anchor the cardiac support system 100 in a blood vessel. The drive unit 125, may be implemented, for example, as a motor or as a motor element that may be configured, for example, to power the sensor device 105 and/or the pump element 150. The pump element 150 may be implemented, for example, as an impeller or as a pump wheel. According to an example, the deployable anchoring structure 140 may be connected to the drive unit 125 by a web 160. With this, the cardiac support system 100 may allow the greatest possible protection of a cable, avoid electrical connections along a surface of the cardiac support system 100, improve functional integrity of the transferred components, e.g., an inlet tube, and ensure that the pump can be manufactured and assembled.

The cardiac support system 100, as described, may include several individual components. During production, for example, a suitable production and assembly sequence of the individual components, also known as sensor components, may be determined. The production and assembly sequence may advantageously adhere to necessary time sequences of, for example, surface treatment steps and simultaneously define interfaces between production partners. Furthermore, the described approach ensures a biocompatible and long-term stable integration of a sensor cable, which may be used as a connection interface in a pump assembly. The connection interface may occupy minimal installation space without reducing pump diameter, thus not impairing efficiency of the pump element 150.

The cardiac support system 100 may include a sensor head, described here as the sensor device 105, an inlet grille with the at least one inlet opening 130, the inlet tube 110, an outlet grille, the pump element 150, at least one outlet opening 135, the deployable anchoring structure 140, which may, for example, include bars coupled to the drive unit 125, and the connection interface 155, which may be configured for merging electrical connection lines and a supply cable.

FIG. 2 shows a schematic representation of a cardiac support system 100 according to an example of an implanted state. The cardiac support system 100 corresponds to or at least resembles the cardiac support system 100 described in FIG. 1 and, according to an example, may be positioned in a heart 200, more precisely across an aortic valve 205 of the heart 200, so that a body fluid from the left ventricle 210 may be aspirated through at least one inlet opening and discharged into the aorta 215 through at least one outlet opening. This may increase a patient's cardiac output per minute, symbolized by an arrow 220, and may thus support or replace the physiological function of the heart 200.

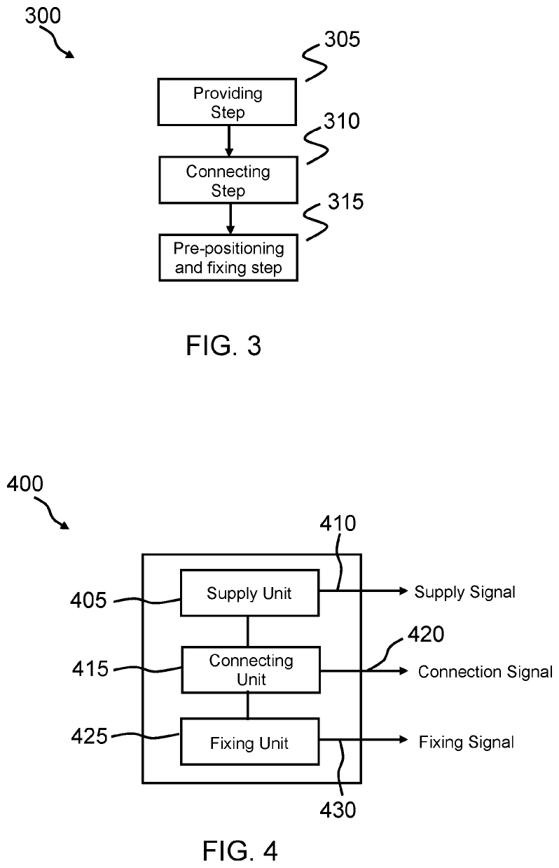

FIG. 3 shows a flowchart of a manufacturing process 300 for manufacturing a cardiac support system according to an example. The manufacturing process 300 describes an example manufacturing process for the cardiac support system 100 as described in, for example, FIGS. 1 and/or 2. The manufacturing process 300 may include a preparation step 305, a joining step 310, and optionally a pre-positioning and fixing step 315. The preparation step 305 may include providing and/or manufacturing a sensor device, an inlet tube with a first end and a second end opposite the first end, and a drive unit for operating the cardiac support system. The inlet tube may be configured to aspirate a patient's body fluid. In the joining step 310, the sensor device may be connected to the first end of the inlet tube and the drive unit may be connected to the second end of the inlet tube to create a result component, which may be a portion of the cardiac support system. According to an example, the second end of the inlet tube may be connected to a component which may be connected to the drive unit. According to an example, the component may include at least one outlet opening. In the optional pre-positioning and fixing step 315, the sensor device, the inlet tube and/or the drive unit may be pre-positioned and connected to each other. In particular, the sensor device, the inlet tube and/or the drive unit may be bonded, siliconized and/or conductively bonded together.

According to an example, in the preparation step 305 of the manufacturing process 300, a housing element may be provided as an option of the cardiac support system, which may be connected to the drive unit in the joining step 310.

Furthermore, according to an example, the joining step 310 may connect the sensor device and the inlet tube to create a connecting device. Optionally, the connecting device may be connected to the drive unit to create a result component, which may be a portion of the cardiac support system.

FIG. 4 shows a block diagram of a device 400 according to an example. The device 400 may be configured, for example, to perform or at least control the manufacturing process for manufacturing a cardiac support system as described in FIG. 3. The device 400 may be equipped with a supply unit 405, which may be configured, for example, by a supply signal 410, to supply a sensor device, an inlet tube having a first end and a second end opposite the first end, the inlet tube being configured to aspirate a patient's body fluid, and a drive unit configured to operate the cardiac support system. The device 400 may be equipped with a connecting unit 415, which may be configured, for example, by a connection signal 420, to cause the sensor device to be connected to the first end of the inlet tube and the drive unit to be connected to the second end of the inlet tube. Optionally, the device 400 may be equipped with a fixing unit 425, which may be configured, for example, by a fixing signal 430, to effect pre-positioning and fixing of the sensor device, the inlet tube and/or the drive unit, where the sensor device, the inlet tube and/or the drive unit may be bonded, siliconized and/or conductively bonded to one another in a fixing step.

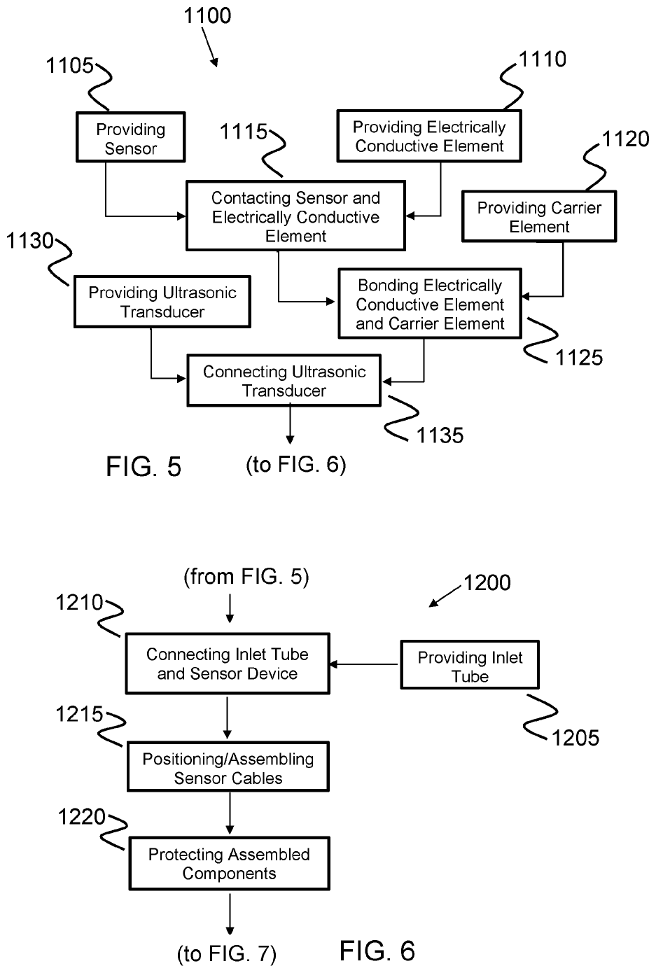

FIG. 5 shows a production sequence 1100 for manufacturing a sensor device with a plurality of sensor components according to an example. The production sequence 1100 represents at least a partial sequence of the manufacturing process 300 as described in FIG. 3. The production sequence 1100 may include a sub-step 1105 of manufacturing and/or providing a sensor and a sub-step 1110 of manufacturing and/or providing at least one electrically conductive element, such as a thin film substrate. In a contacting sub-step 1115, the sensor and the at least one electrically conductive element may be electrically contacted with each other. In a providing sub-step 1120, a carrier element, such as Polyether ether ketone (PEEK), may be provided and then may be coupled and/or bonded to the sensor—electrically conductive element combination or, for example, siliconized in a bonding sub-step 1125. According to an example, an ultrasonic transducer may be additionally provided in sub-step 1130, which may be connected to the result component from the bonding sub-step 1125, for example, in a conductive bonding sub-step 1135 to obtain the sensor device as described in, for example, FIGS. 1 and/or 2.

Thus, sub-steps 1105, 1110, 1115, 1120, 1125, 1130, and 1135 describe manufacturing of a sensor device. In sub-step 1105, at least one sensor may be manufactured and/or provided in an electrically contactable form, for example, on a substrate or sub-mount. In sub-step 1110, at least one electrically conductive element may be manufactured and/or provided, for example, in the form of a cable or an electrical thin-film substrate, for electrically connecting the at least one sensor in a distal tip with an electrical connection interface. Contacting sub-step 1115 may include electrical contacting of the at least one sensor to the at least one electrically conductive element. In providing sub-step 1120, a carrier element, for example, PEEK, may be manufactured and/or provided by milling or injection molding. In bonding sub-step 1125, the at least one electrically conductive element with the at least one electrically connected sensor may be bonded to the carrier element with subsequent encapsulation of a sensor cavity in the carrier element with medical-grade silicone. The at least one electrically conductive element may protrude from a sensor head to be, for example, subsequently connected further along the cardiac support system. At sub-step 1130, the ultrasonic transducer such as a piezo disk or a structure of a transducer layering including backing and matching layers, may be manufactured and/or provided and may be encapsulated with a biocompatible material such as medical-grade silicone. In conductive bonding sub-step 1135, the ultrasonic transducer may be conductively bonded into a sensor package on the at least one electrically conductive element and an ultrasonic lens may be applied, for example, injection molded, to form the sensor device.

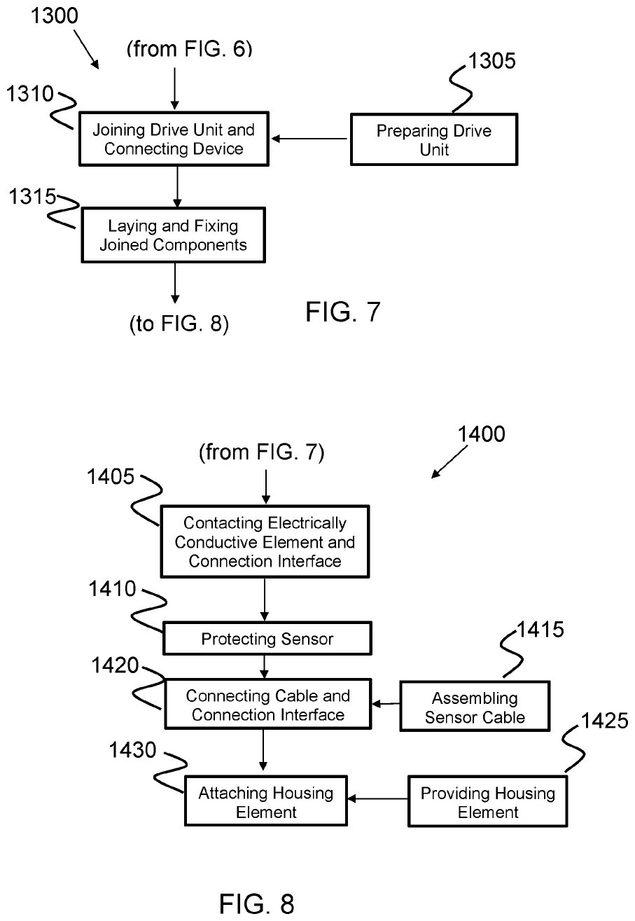

FIG. 6 shows an assembly sequence 1200 for manufacturing a connecting device according to an example. The assembly sequence 1200 represents at least a partial sequence of the manufacturing process 300 as described in FIG. 3. In sub-step 1205, an inlet tube may be provided and/or manufactured, which may be connected in sub-step 1210 with a sensor device, for example, as made in FIG. 5. Corresponding sensor cables may be assembled according to an example in sub-step 1215, where the sensor cables may be made and/or joined to the connecting device.

According to an example, in a siliconizing sub-step 1220, assembled components may be siliconized, for example, by a coater, to obtain the connecting device as described in FIG. 1. A connecting device assembly sequence is described according to an example using sub-steps 1205, 1210, 1215, and 1220. In sub-step 1205, for example, an inlet tube may be manufactured by laser cutting from a Nitinol tube or by braiding Nitinol wires. In sub-step 1210, for example, a previously manufactured sensor device may be joined to an inlet grille of the inlet tube (also known as inlet cage) by press fitting, supported by a glued joint. In sub-step 1215, at least one electrically conductive element may be positioned along the inlet tube. In some examples, the at least one electrically conductive element may be positioned in or in alignment with a spiral structure of the inlet tube and/or a fixation of a carrier element along the spiral structure by biocompatible adhesive. In some examples, perforations in the inlet tube, which may result in the inlet tube not being liquid tight, may be sealed in sub-step 1220.

Sub-step 1220 may include closure of perforations with a biocompatible polymer, for example, a silicone coating. In this step, the pre-fixed at least one electrically conductive element may be enclosed between the inlet tube and the silicone coating and thus mechanically protected.

FIG. 7 shows a further assembly sequence 1300 for connecting a connecting device to a drive unit according to an example. The assembly sequence 1300 represents at least a partial sequence of the joining step 310 as described in FIG. 3, and builds, for example, on the assembly sequence 1200 as described in FIG. 6, for example following sub-step 1220. In preparation sub-step 1305, a drive unit may be prepared (e.g., provided and/or manufactured), which then may be assembled with a connecting device in a joining sub-step 1310. In a laying and fixing sub-step 1315, the joined components may be glued together, for example, to make a result component of a cardiac support system.

In other words, according to an example, sub-steps 1305, 1310, and 1315 describe a motor assembly, which may include the drive unit. In sub-step 1305, the drive unit may be manufactured and/or provided in parallel to the production sequence 1100 and/or the assembly sequence 1200. In sub-step 1310, the drive unit from sub-step 1305 and the connecting device with at least one electrically conductive element from sub-step 1220 may be joined together, for example, by press fitting with supported bonding (e.g., gluing, laser welding, and/or riveting). In sub-step 1315, a remaining excess length of the at least one electrically conductive element protruding from an inlet tube may be guided along the drive unit to a connection interface and fixed to a surface of a cardiac support system with a biocompatible adhesive. Then, surface protective coating of a cable may be carried out, for example, using biocompatible epoxy resin.

FIG. 8 shows a cabling sequence 1400 for wiring a cardiac support system according to an example. According to this example, the cabling sequence 1400 follows the further assembly sequence 1300 as described in FIG. 7, for example, following step 1315. In contacting sub-step 1405, a sensor cable of a cardiac support system may be electrically connected with a connection point in a connection interface. Subsequently, in protecting sub-step 1410, a sensor positioned on or near the connection interface may be protected by a biocompatible protective cover such as silicone. The cabling sequence 1400 may include a sleeve assembly sub-step 1415, where a sleeve may be assembled over the sensor cable to obtain a hybrid cable. The hybrid cable may be electrically connected to the connection interface in connecting sub-step 1420. In sub-step 1425, a housing element may be provided and/or manufactured, which, in attachment sub-step 1430, may be attached to the components from sub-step 1420.

In other words, components may be assembled together at the connection interface by sub-steps 1405, 1410, 1415, 1420, 1425, and 1430. In sub-step 1405, at least one electrically conductive element may be electrically connected to metallic pins of the connection interface by, for example, conductive bonding or soldering. In sub-step 1410, sensors in an area of the connection interface or the drive unit may be covered with a protective covering, for example, encapsulated with silicone. In sub-step 1415, the sensor cable may be assembled, and sleeves for contacting cable strands to the pins of the connection interface may be connected to the cable. In sub-step 1420, the sensor cable may be electrically connected to the connection interface, for example, to pins of the connection interface, for example, by welding, soldering, and/or conductive bonding. A protective housing and/or strain relief may be provided and/or manufactured, for example, from a titanium sheet, in sub-step 1425. In sub-step 1430, electrical connection points in the connection interface may be connected and coated, for example, with a biocompatible epoxy resin or silicone. After coating, the protective housing from sub-step 1425 may be assembled to cover the connection interface. Finally, a protective coating may be applied on the entire system, for example, with Parylene C.

If an execution example includes an “and/or” link between a first feature and a second feature, this should be read in such a way that the execution example has both the first feature and the second feature according to one execution form and either only the first feature or only the second feature according to another execution form.

Various modifications to the implementations described in this disclosure will be readily apparent to those skilled in the art, and the generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “example” is used exclusively herein to mean “serving as an example, instance, or illustration.” Any implementation described herein as “example” is not necessarily to be construed as preferred or advantageous over other implementations, unless otherwise stated.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Additionally, other implementations are within the scope of the following claims. In some cases, the actions recited in the claims can be performed in a different order and still achieve desirable results.

It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

1. A method for manufacturing a cardiac support system, the method comprising the steps of:
providing a sensor device, an inlet tube with a first end and a second end opposite the first end, the inlet tube configured to aspirate a body fluid of a patient, and a drive unit for operating the cardiac support system; and
connecting the sensor device with the first end of the inlet tube and connecting the drive unit with the second end of the inlet tube.

2. The method according to claim 1, wherein connecting the sensor device with the first end of the inlet tube forms a connecting device.

3. The method according to claim 2, wherein connecting the connecting device and the drive unit forms at least a portion of the cardiac support system.

4. The method according to one of the preceding claim 1, further comprising: providing a housing element; and connecting step the drive unit to the housing element.

5. The method according to claim 1, further comprising:
pre-positioning and fixing the sensor device, the inlet tube and the drive unit, wherein the sensor device, the inlet tube and the drive unit are connected to one another.

6. The method according to claim 2, further comprising:
providing a sensor;
providing at least one electrically conductive element; and
electrically contacting the sensor and the at least one electrically conductive element.

7. The method according to claim 6, further comprising manufacturing the sensor device by:
providing a carrier element;
bonding the carrier element to the electrically contacted sensor and the at least one electrically conductive element;
providing an ultrasonic transducer; and
conductively bonding the ultrasonic transducer to a result component from the bonding step.

8. The method according to claim 7, further comprising manufacturing the connecting device by:
providing the inlet tube; and
connecting the inlet tube to the manufactured sensor device.

9. The method according to claim 8, further comprising:
assembling corresponding sensor cables and/or joining the sensor cables to the connecting device; and
siliconizing assembled components.

10. The method according to claim 8, further comprising assembling the drive unit by:
providing the drive unit;
joining the drive unit to the connecting device; and
laying and fixing the joined drive unit and connecting device.

11. The method according to claim 10, further comprising:
electrically contacting a sensor cable of the cardiac support system with a connection point in a connection interface; and
protecting a sensor positioned on or near the connection interface by a biocompatible cover.

12. The method according to claim 11, further comprising:
assembling a sleeve over the sensor cable to obtain a hybrid cable;
electrically connecting the hybrid cable to the connection interface;
providing the housing element; and
attaching the housing element to a result component from the electrically connecting step.

13. A device configured to execute and/or control the steps of the method according to claim 5.

14. A computer program configured to execute and/or control the steps of the method according to claim 5.

15. (canceled)

16. A cardiac support system comprising:
a sensor device;
an inlet tube with a first end and a second end opposite the first end coupled to the sensor device, the inlet tube configured to aspirate a body fluid of a patient; and
a drive unit for operating the cardiac support system, wherein the drive unit is connected to the second end of the inlet tube.

17. The cardiac support system according to claim 16, wherein the inlet tube comprises at least one inlet opening at the first end, wherein the at least one inlet opening is configured:
to allow the body fluid to flow into the inlet tube, and
as an interface between the sensor device and the inlet tube.

18. The cardiac support system according to claim 16, wherein the inlet tube further comprises at least one outlet opening at the second end, wherein the at least one outlet opening is configured to enable the body fluid to be discharged from the inlet tube.

19. The cardiac support system according to claim 16, wherein the drive unit comprises a deployable anchoring structure, a housing element, a pump element and a connection interface.

20. The cardiac support system according to claim 16, wherein the sensor device comprises a plurality of sensor components, the plurality of sensor components comprising at least one sensor, a layer substrate, a carrier element and/or an ultrasonic transducer.

21. The cardiac support system according to claim 20, wherein at least two of the plurality of sensor components are electrically connected to each other.
